(19) United States
(12) Patent Application Publication
(10) Pub. No.: US 2014/0213496 A1
(43) Pub. Date: Jul. 31, 2014

(54) NOVEL METHOD FOR IMPROVING THE FOAMING PROPERTIES OF CLEANING COMPOSITIONS FOR TOPICAL USE

(71) Applicant: Emmanuelle Merat, Lautrec (FR)

(72) Inventor: Emmanuelle Merat, Lautrec (FR)

(73) Assignee: SOCIETE D'EXPLOITATION DE PRODUITS POUR LES INDUSTRIES CHIMIQUES SEPPIC, Paris (FR)

(21) Appl. No.: 14/239,848

(22) PCT Filed: Aug. 24, 2012
(86) PCT No.: PCT/FR2012/051931

§ 371 Date: Feb. 20, 2014

(30) Foreign Application Priority Data
Aug. 31, 2011 (FR) 1157699

(51) Int. Cl. A61K 8/81; A61Q 1/14; A61Q 19/10

(52) U.S. Cl. CPC A61K 8/817; A61Q 19/10; A61Q 1/14
USPC 510/136; 510/130

(57) ABSTRACT

Method for improving foaming properties of cleaning compositions for topical use, having a 4.0≦pH≦6.5, and including at least one foaming surfactant, includes incorporating an effective quantity of a cross-linked anionic polyelectrolyte into the cleaning composition, the cross-linked anionic polyelectrolyte being obtained through polymerization, in the presence of at least one cross-linking agent, of at least one monomer having a strong acid function, the monomer being 2-methyl 2-[(1-oxo 2-propenyl)amino]1-propanesulfonic acid, which is partially or completely salified, with at least one neutral monomer selected from the N,N-dialkyl acrylamides, wherein each of the alkyl groups includes between one and four carbon atoms and at least one monomer of formula (I), in which R is a linear or branched alkyl radical including eight to twenty carbon atoms, and n is a number greater than or equal to one and less than or equal to twenty.

The present invention relates to the field of the cosmetic and/or pharmaceutical industry. Its subject matter is a novel method for improving the foaming properties of cleaning and/or foaming formulations, novel compositions and methods for preparing same.

Cleaning formulae for the face, body and hair, and more generally body and hair hygiene products presented in the form of shampoos, lotions, gels or liquid soaps, require the formation of foam during application thereof to the part of the body to be cleansed. This is because, in the mind of the consumer, the formation of foam constitutes one of the proofs of the efficacy of the cleaning. The volume of this foam, the stability thereof, and the agreeable sensations that it causes, are important parameters to be taken into account for hoping for the commercial success of these formulations.

To do this, these cleaning formulations comprise cleaning and foaming surfactants, whether they be of cationic, anionic, amphoteric or non-ionic in nature.

As the surface of the skin has a weakly acid pH value, generally between 4.0 and 6.5 (with the exception of fatty skins, which have a pH above 6.5), the cleaning formulations must have a pH of the same order in order not to disrupt the pH of the skin and confer a fatty character thereon.

Formulations for cleaning the face, body and hair must have sufficient consistency for their application on the surface of the skin to be cleansed to be effective. This is why they often comprise thickening agents compatible with the cleaning and foaming surfactants, which give them this consistency.

Among the thickeners normally used for thickening these formulations, there is sodium chloride; at an optimum dose, it causes a formation of surfactant micelles, which reduce the movements of the fluid and then increase the viscosity thereof. This thickening means is inexpensive. However, the foam during washing is not very stable and the size of the air bubbles making it up is great, which, during cleaning of the face, results in increasing the risk of contact with the eyes and therefore ocular irritation, resulting in a stinging of the eyes that is uncomfortable for the consumer.

Hydrocolloids of plant or biosynthetic origin, such as for example gum xanthan, gum karaya, carrageenates, alginates and galactomannans, are also thickening agents frequently used in the cosmetic industry, since the thickening that they cause is insensitive to the presence of the electrolytic species contained in skin cleaning formulations. The texture of these formulations is however “free-running”, in that they adhere excessively to hard surfaces and to the skin, which is a drawback, both on conditioning chains since they cause an increase in the washing time of the equipment used, and when gripping by the consumer before application to the skin.

Crosslinked anionic copolymers based on methacrylic acid or acrylic acid, or methacrylic acid or acrylic acid esters, optionally hydrophilically modified, prepared by polymerisation in direct emulsion, are also used in cosmetics. They are respectively known to persons skilled in the art by the names “alkaline swellable emulsion” (or “ASE”) and “hydrophobically alkaline swellable emulsion (or “HASE”). Thickening agents of the “HASE” type are described in the international application published on 2 May 2002 under the number W02002/34793 A2. The thickening efficacy thereof is however satisfactory only as from a pH greater than or equal to 6.5. The texture of the formulations is of the “gelled” type, which in this case also is a drawback when gripping by the consumer before application to the skin.

Persons skilled in the art also know about the use of crosslinked or branched anionic polyelectrolytes, which are crosslinked and/or branched homopolymers or copolymers of hydrosoluble unsaturated monomers, such as for example acrylic acid or derivatives of acrylic acid, acrylamide, or derivatives of acrylamide, acrylamidomethyl propanesulfonic acid marketed under the names CARBOPOL™, ULTREZ™ 10, PEMULEN™ TR1, PEMULEN™ TR2, SIMULGEL™ EG, SIMULGEL™ EPG, LUVIGEL™ EM, SALCARE™ SC91, SALCARE™ SC92, SALCARE™ SC95, SALCARE™ SC96, FLOCARE™ ET100, FLOCARE™ ET58, HISPAGEL™, SEPIGEL™ 305, SEPIGEL™ 501, SEPIGEL™ 502, SIMUGEL™ NS, SIMUGEL™ 800, SIMUGEL™ 600, SIMUGEL™ A, SEPIPLUS™ 250, SEPIPLUS™ 265, SEPIPLUS™ 400, SEPINOV™ EMT 10, NOVEMER™ EC1, ARISTOFLEX™ AVC, ARISTOFLEX™ HBM, RAPITHIX™ A60, RAPITHIX™ A100, COSMEDIA SP and STABILEZE™ 06. These crosslinked or branched anionic polyelectrolytes are in the form of inverse latexes, obtained by radical polymerisation into inverse emulsion, or in the form of powders, obtained by precipitating polymerisation or by atomisation of inverse latexes. The use thereof as a thickening agent does not however prove to be sufficiently effective to thicken in the presence of the electrolytic species contained in skin cleaning formulations.

Crosslinked or branched ionic polyelectrolytes such as those described in the international patent application published under the number WO 2011/030044, effectively thicken skin cleaning formulations, but use thereof at a pH greater than or equal to 7.0 in the presence of foaming surfactants does not however make it possible to obtain a sufficiently fine foam.

The American U.S. Pat. No. 7,025,973 B2 describes copolymers obtained by radical polymerisation of 2-methyl 2-[(1-oxo 2-propenyl)amino]1-propanesulfonic acid with unsaturated olefinic monomers, comprising at least one oxygen or nitrogen atom, with a molecular weight of less than 500 g/mol, and at least one macromer. The American U.S. Pat. No. 7,025,973 B2 does not disclose cosmetic compositions comprising crosslinked or branched anionic polyelectrolytes, as described in the international application published under the number WO 2011/030044, and foaming surfactants, nor the use of said crosslinked or branched anionic polyelectrolytes for preparing cleaning cosmetic compositions generating a small volume of foam, so as to limit the risk of contact of said foam with the eyes.

However, in the particular case of cleaning of the face, which aims to cleanse the pores of the skin, or to eliminate impurities, excess sebum, dead cells or traces of makeup from the skin, it is preferable for the cleaning formulations to generate a small volume of foam and for the air bubbles that make up this foam to be stable and have a sufficiently fine average size during the cleaning phase, so as to limit the risk of contact with the eyes and therefore ocular irritation resulting in stinking of the eyes that is uncomfortable for the consumer.

In order to attempt to overcome with these drawbacks, the inventors have therefore sought to develop a novel solution for having a cleaning formulation that has a pH greater than or equal to 4.0 and less than or equal to 6.5, more particularly greater than or equal to 6.0 and less than or equal to 4.5, which is easy to grip by the consumer, and which generates a fine stable foam during use thereof in the operation of the cleaning the skin, and more particularly the face.

This is why, according to a first aspect, the subject matter of the invention is a method for improving the foaming properties of a cleaning formulation for topical use, with a pH greater than or equal to 4.0 and less than or equal to 6.5 and comprising at least one foaming surfactant, said method being characterised in that an effective quantity of a crosslinked anionic polyelectrolyte (P) derived from polymerisation is incorporated in said cleaning formulation for topical use, in the presence of at least one crosslinking agent, at least one monomer having a strong acid function, said monomer being 2-methyl 2-[(1-oxo 2-propenyl)amino]1-propanesulfonic acid, partially or completely salified, with at least one neutral monomer chosen from the N,N-dialkyl acrylamides, in which each of the alkyl groups comprise between one and four carbon atoms, and at least one monomer of formula (I):

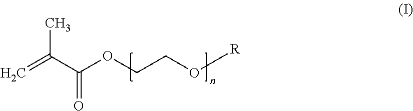

in which R represents a linear or branched alkyl radical comprising from eight to twenty carbon atoms and n represents a number greater than or equal to one and less than or equal to twenty.

Foaming surfactant means, in the definition of the method as defined above, any foaming surfactant whether it be ionic, cationic, amphoteric or non-ionic, that is topically acceptable.

Effective quantity of a crosslinked anionic polyelectrolyte (P) means a quantity such that:
the mean diameter of at least 70% of the air bubbles present in the foam formed by the cleaning formulation is less than or equal to 150 micrometres (μm) after a period of 10 minutes as from the moment of generation of said foam;
the mean diameter of no more than 30% of the air bubbles present in the foam formed by the cleaning formulation is greater than or equal to 150 μm and less than or equal to 450 μm after a period of 10 minutes as from the moment of generation of said foam;
and such that the viscosity of the cleaning formulation prepared is greater than or equal to 2,000 mPa·s and less than or equal to 30,000 mPa·s, more particularly greater than or equal to 2,000 mPa·s and less than or equal to 20,000 mPa·s, measured at a temperature of 20° C. by means of a viscometer of the Brookfield LVT type at a speed of 6 revolutions/minute.

The expression “for topical use” used in the definition of the method as defined above means that said cleaning formulation for topical use is used by application on the skin of the body and face, on the hair, on the scalp or on the mucous membranes, whether it be a direct application in the case of a cosmetic, dermocosmetic, dermo-pharmaceutical or pharmaceutical formulation or indirect application for example in the case of a body hygiene product in the form of a textile or paper wipe or sanitary products intended to be in contact with the skin or mucous membranes.

Crosslinked anionic polyelectrolyte means, in the definition of the method as defined above, a non-linear crosslinked anionic polyelectrolyte in the state of a three-dimensional lattice insoluble in water, but swellable in water and therefore leading to the obtaining of a chemical gel.

Partially salified or completely salified means, in the definition of the method as defined above, that said strong acid function of the monomer incorporating it is, partially or completely salified, generally in the form of an alkaline metal salt, for example sodium salt or potassium salt, or in the form of an ammonium salt.

In the context of the method as described above and the subject matter of the present invention, said crosslinked anionic polyelectrolyte (P) used generally comprises between 5% molar and 95% molar of the strong acid function monomer, more particularly between 10% molar and 90% molar, especially between 20% molar and 80% molar and more especially between 60% molar and 80% molar.

In the context of the method that is the subject matter of the present invention, said crosslinked anionic polyelectrolyte (P) as previously defined comprises more particularly between 4.9% molar and 90% molar of neutral monomer chosen from the N,N-dialkyl acrylamides, wherein each of the alkyl groups comprises between one and four carbon atoms, more particularly between 9.5% molar and 85% molar, especially between 15% molar and 75% molar, and more especially between 15% molar and 39.5% molar.

In said crosslinked anionic polyelectrolyte (P) used in the method that is the subject matter of the present invention and as defined above, the strong acid function of the monomer incorporating same is in particular the sulfonic acid function, partially or completely salified.

According to a particular aspect of the method as defined above, effective quantity of a crosslinked anionic polyelectrolyte (P) means, for 100% by mass of the cleaning formulation, a proportion by mass of said anionic polyelectrolyte (P) lying between 0.1% and 2.0%, and more particularly between 0.1% by mass and 1.5% by mass, even more particularly between 0.1% by mass and 1% by mass.

The neutral monomer chosen from the N,N-dialkyl acrylamides, wherein each of the alkyl groups comprises between one and four carbon atoms, is in particular chosen from N,N-dimethyl acrylamide, N,N-diethyl acrylamide and N,N-dipropyl acrylamide.

In the context of the present invention, said crosslinked anionic polyelectrolyte (P) as defined previously comprises between 0.1% molar and 10% molar of monomers of formula (I) and more particularly between 0.5% molar and 5% molar of monomers of formula (I).

In formula (I) of the monomer present in said crosslinked anionic polyelectrolyte (P) used in the method that is the subject matter of the present invention, linear or branched alkyl radical comprising from eight to twenty carbon atoms means more particularly for R:
either a radical derived from linear primary alcohols such as for example the octyl, decyl, undecyl, dodecyl, tridecyl, tetradecyl, pentadecyl, hexadecyl, heptadecyl, octadecyl, nonadecyl or eicosyl radical;
or a radical derived from Guerbet alcohols, which are branched 1-alkanols complying with the general formula:

CH₃—(CH₂)ₚ—CH[CH₃—(CH₂)_{P-2}]—CH₂OH

wherein p represents an integer number between 2 and 9, such as, for example, the 2-ethyl hexyl, 2-propyl heptyl, 2-butyl octyl, 2-pentyl nonyl, 2-hexyl decyl or 2-octyl dodecyl radicals;
or a radical derived from the isoalkanols complying with the general formula:

CH₃—CH(CH₃)—(CH₂)ₘ—CH₂OH

wherein m represents an integer number between 2 and 16, such as, for example, the 4-methyl pentyl, 5-methyl hexyl, 6-methyl heptyl, 15-methyl pentadecyl or 16-methyl heptadecyl radicals, or the 2-hexyl octyl, 2-octyl decyl or 2-hexyl dodecyl radicals.

According to a particular aspect, the subject matter of the invention is a method as described previously, characterised in that said crosslinked anionic polyelectrolyte (P) comprises, for 100% molar:
from 20% molar to 80% molar of monomeric units derived from the monomer comprising a partially or completely salified strong acid function;
from 15% molar to 75% molar of monomeric units derived from a neutral monomer chosen from the N,N-dialkyl acrylamides, wherein each of the alkyl groups comprises between one and four carbon atoms;
from 0.5% molar to 5% molar of monomeric units derived from a monomer of formula (I) as defined previously.

According to another particular aspect of the present invention, the subject matter thereof is a method as described previously, characterised in that, in said crosslinked anionic polyelectrolyte (P), said monomer having a strong acid function is 2-methyl, 2-[(1-oxo 2-propenyl)amino]1-propanesulfonic acid partially salified or completely salified and more particularly 2-methyl 2-[(1-oxo 2-propenyl)amino]1-propanesolufonic acid partially salified or completely salified in the form of alkaline metal salt, such as for example sodium salt or potassium salt, or in the form of ammonium salt.

According to a particular aspect of the present invention, the subject matter thereof is the method as described previously, characterised in that, in said crosslinked anionic polyelectrolyte (P), said neutral monomer is N,N-dimethyl acrylamide.

According to a particular aspect of the present invention, the subject matter thereof is the method as described previously, characterised in that, in said crosslinked anionic polyelectrolyte (P) and for said monomer of formula (I) as defined previously, R represents an alkyl radical comprising 12 to 18 carbon atoms.

According to another particular aspect, the subject matter of the invention is the method as defined previously, characterised in that, in said crosslinked anionic polyelectrolyte (P) and for said monomer of formula (I) as defined previously, N represents an integer number between 3 and 20.

According to an even more particular aspect, the subject matter of the invention is the method as defined previously, characterised in that, in said crosslinked anionic polyelectrolyte (P), said monomer of formula (I) is tetraethoxylated lauryl methacrylate.

According to an even more particular aspect, the subject matter of the invention is the method as defined previously for which the monomer of formula (I) included in the crosslinked anionic polyelectrolyte (P) is eicosaethoxylated stearyl methacrylate.

According to another particular aspect, the subject matter of the invention is the method as defined previously for which said crosslinked anionic polyelectrolyte (P) is crosslinked with a diethylenic or polyethylenic compound in the molar proportion expressed with respect to the monomers used of 0.005% to 1%, more particularly from 0.01% to 0.5% and especially from 0.01% to 0.25%. The crosslinking agent is particularly chosen from ethylene glycol dimethacrylate, tetraallyloxyethane, ethylene glycol diacrylate, diallyl urea, triallyl amine, trimethylolpropane triacrylate or methylene-bis(acrylamide) or a mixture of these compounds.

The crosslinked anionic polyelectrolyte (P) used in the method that is the subject matter of the present invention may also comprise various additives, such as complexing agents, transfer agents or chain-limiting agents.

According to a particular aspect, the subject matter of the invention is a method as described previously for which said crosslinked anionic polyelectrolyte (P) is chosen from the terpolymers of 2-methyl 2-[(1-oxo 2-propenyl)amino]1-propanesulfonic acid partially salified in ammonium form, N,N-dimethyl acrylamide and tetraethoxylated lauryl methacrylate, crosslinked with trimethylol propanetriacrylate or the terpolymers of 2-methyl 2-[(1-oxo 2-propenyl)amino]1-propanesulfonic acid partially salified in the form of ammonium salt, N,N-dimethyl acrylamide and eicosaethoxylated stearyl methacrylate, crosslinked with trimethylol propanetriacrylate.

According to an even more particular aspect, the subject matter of the invention is a method as described previously for which said crosslinked anionic polyelectrolyte (P) is a terpolymer of 2-methyl 2-[(1-oxo 2-propenyl)amino]1-propanesulfonic acid partially salified in ammonium form, N,N-dimethyl acrylamide and tetraethoxylated lauryl methacrylate, crosslinked with trimethylol propanetriacrylate.

According to an even more particular aspect, the subject matter of the invention is a method as described previously for which said crosslinked anionic polyelectrolyte (P) comprises, for 100% molar:
from 60% molar to 80% molar of monomeric units derived from 2-methyl 2-[(1-oxo 2-propenyl)amino]1-propanesulfonic acid partially salified in ammonium form;
from 15% molar to 39.5% molar of monomeric units derived from N,N-dimethyl acrylamide; and
from 0.5% molar to 5% molar of monomeric units derived from tetraethoxylated lauryl methacrylate.

In the method that is the subject matter of the present invention as defined above, among the anionic surfactants that can be associated with the crosslinked anionic polyelectrolyte (P) in the cleaning formulations for topical use and having a pH greater than or equal to 4.0 and less than or equal to 6.5, mention can be made particularly of alkaline metal salts, alkaline-earth metal salts, ammonium salts, amine salts and aminoalcohol salts of the following compounds: alkylether sulfates, alkylsulfates, alkylamidoether sulfates, alkylaryl polyether sulfates, monoglyceride sulfates, alpha-olefin sulfonates, paraffin sulfonates, alkylphosphates, alkylether phosphates, alkyl sulfonates, alkylamido sulfonates, alkylaryl sulfonates, alkylcarboxylates, alkyl sulfocuccinates, alkylether sulfosuccinates, alkylamide sulfo succinates, alkyl sulfoacetates, alkylsarcosinates, acyl isethionates, N-acyltaurates and acyllactylates. Among anionic surfactants, mention can also be made of lipoamino acids, lipoproteins, lipopeptides, derivatives of lipoproteins, derivatives of proteins, fatty acid salts and acid salts of copra oil, optionally hydrogenated.

According to a more particular aspect of the present invention, the subject matter of the latter is a method as described previously, for which the cleaning formulation for topical use comprises at least one anionic foaming surfactant of formula (II):

R₂—O—(CH₂—CH₂—O)ₚSO₃—X (II)

in which R₂ represents an aliphatic hydrocarbon radical, saturated or unsaturated, linear or branched, comprising 6 to 22 carbon atoms, p represents a decimal number between 1 and 10, preferably between 2 and 4, and X represents the cation of an alkaline metal or alkaline-earth metal, the ammonium ion, the hydroxyethyl ammonium ion, the tris(hydroxyethyl) ammonium ion or a mixture of compounds of formula (II).

In formula (II) as defined above, X represents for example sodium, magnesium or the ammonium ion.

In the method that is the subject matter of the present invention and as defined above, among the amphoteric surfactants that can be associated with the crosslinked anionic polyelectrolyte (P) in the cleaning formulations for topical use and having a pH greater than or equal to 4.0 and less than or equal to 6.5, mention will be made particularly of the alkylbetaines, alkylamido betaines, sultaines, alkylamido alkylsulfo betaines, derivatives of imidazolines, phosphobetaines, amphopolyacetates and amphopropionates.

According to a more particular aspect of the present invention, the subject matter of the latter is a method as described previously for which the cleaning formulation for topical use comprises at least one amphoteric foaming surfactant of formula (III):

R₃—C(O)—NH(CH₂)_{q}—N⁺(R₄)(R₅)—(CH₂)ₛ—CO₂⁻ (III)

in which R₃ represents an aliphatic hydrocarbon radial, saturated or unsaturated, linear or branched, comprising 7 to 21 carbon atoms, R₄ and R₅ represent independently of each other an aliphatic radical, saturated or unsaturated, linear or branched, optionally substituted with a hydroxyl group, comprising 1 to 4 carbon atoms, q represents an integer number between 2 and 6, and s represents an integer number equal to 1 or 2, or a mixture of compounds of formula (III).

In formula (III) as defined above, R₃—C(O)— represents for example the octanoyl radical, the decanoyl radical, the lauroyl radical or the cocoyl radical.

In formula (III) as defined above, q is for example equal to 3.

In formula (III) as defined above, R₄ and R₅ represents a methyl radical.

According to a more particular aspect of the present invention, the subject matter thereof is a method as defined previously for which the amphoteric foaming surfactant of formula (III) is cocamidopropryl betaine.

According to another particular aspect of the present invention, the subject matter thereof is a method as described previously, for which the cleaning formulation for topical use comprises a mixture of at least one compound of formula (II), as defined previously, with at least one compound of formula (III) as defined previously.

In the method that is the subject matter of the present invention and as defined above, among the non-ionic surfactants that can be associated with the crosslinked anionic polyelectrolyte (P) in the cleaning formulations for topical use and having a pH greater than or equal to 4.0 and less than or equal to 6.5, mention will be made particularly of the ethoxylated derivatives of fatty alcohols comprising 8 to 16 carbon atoms, the ethoxylated derivatives of fatty acids comprising 8 to 16 carbon atoms, the ethoxylated derivatives of fatty esters comprising 8 to 16 carbon atoms, the ethoxylated derivatives of monoglycerides comprising 8 to 16 carbon atoms, the ethoxylated derivates of sorbitan, the ethoxylated derivates of mannitan, the alkylpolyglycosides, the derivatives of ricin oil, polysorbates, copra amides, N-alkylamines and amine oxides.

Among the foaming non-ionic surfactants cited above, which are non-ionic surfactants, there are more particularly the compounds of formula (IV):

R₆—O—(S)_{y}—H (IV)

in which y represents a decimal number between 1 and 5, S represents the remainder of a reducing sugar and R₆ represents a linear or branched alkyl radical, saturated or unsaturated, having 8 to 16 carbon atoms, preferably 8 to 14 carbon atoms, or a mixture of compounds of formula (IV).

In the definition of formula (IV) as defined previously, y is a decimal number that represents the average degree of polymerisation of the remainder S. When y is an integer, (S)_{y} is the polymeric remainder of rank y of the remainder S. When y is a decimal number, formula (IV) represents a mixture of compounds: a₁ R₆—O—S—H+a₂ R₆—O—(S)₂—H+a₃ R₆—O—(S)₃—H+ . . . +a_{q} R₆—O—(S)_{q}—H with q representing an integer number between 1 and 10 and in the molar proportions a₁, a₂, a₃, a_{q} such that:

$${\sum }_{q = 10}^{q = 1} {a}_{q} = 1 ; {a}_{1} > 0$$

In the method that is the subject matter of the present invention and as defined above, in the definition of the compounds of formula (IV), y is between 1.05 and 5, and more particularly between 1.05 and 2.

In formula (IV) as defined above, R₆ represents for example the n-octyl radical, the n-decyl radical, the n-dodecyl radical, the n-dodecyl radical or the n-tetradecyl radical.

Reducing sugar means, in the definition of formula (IV), the saccharide derivatives that do not have in their structures any glycoside bond established between an anomeric carbon and the oxygen of an acetal group as defined in the reference work “Biochemistry”, Daniel Voet/Judith G. Voet, p. 250, John Wiley & Sons, 1990. The oligomeric structure (S)_{y} may be in any form of isomerism, whether it be optical isomerism, geometrical isomerism or position isomerism; it may also represent a mixture of isomers.

In the formula (IV) as defined above, the R₆—O— group is bonded to S by the anomeric carbon of the saccharide remainder, so as to form an acetal function.

According to a more particular aspect of the present invention, the subject matter thereof is a method as defined previously for which the cleaning formulation for topical use comprises at least one compound of formula (IV) in which y is a decimal number between 1.05 and 2, S represents the remainder of a reducing sugar chosen from glucose, xylose or arabinose and R₆ represents a radical chosen from the n-octyl, n-decyl, n-dodecyl, n-tetradecyl or n-hexadecyl radicals, or a mixture of compounds of formula (IV).

According to another particular aspect of the present invention, the subject matter thereof is a method as defined previously, characterised in that the ratio by mass between foaming surfactant and crosslinked anionic polyelectrolyte (P) is between 1/10 and 40/1, more particularly between 1/1 and 40/1 and even more particularly between 4/1 and 40/1.

Another subject matter of the invention is a composition (C1) characterised in that it comprises, for 100% of its mass:
from 0.05% to 2% by mass of at least one crosslinked anionic polyelectrolyte (P) derived from the polymerisation of at least one monomer having a strong acid function, said monomer being 2-methyl 2-[(1-oxo 2-propenyl)amino]1-propanesulfonic acid partially or totally salified, with at least one neutral monomer chosen from the N,N-dialkyl acrylamides, wherein each of the alkyl groups comprises between one and four carbon atoms, and at least one monomer of formula (I):

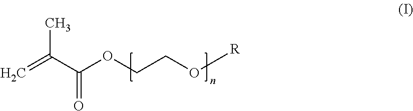

in which R represents a linear or branched alkyl radical comprising from eight to twenty carbon atoms and n represents a number greater than or equal to one and less than or equal to twenty in the presence of at least one crosslinking agent;
from 10% to 50% by mass of at least one foaming surfactant selected from elements of the group formed by the anionic surfactants, non-anionic surfactants and amphoteric surfactants.
from 0.01% to 10% by mass of at least one acid agent (A) selected from the group consisting of the α-hydroxy acids and β-hydroxy acids, free, partially or completely salified.
from 89.94% to 38% by mass of water,
   and in that the pH thereof is greater than or equal to 4.0 and less than or equal to 6.5.

According to a particular aspect, in the composition (C1) that is the subject matter of the present invention, in the polyelectrolyte (P) as defined previously, the neutral monomer is chosen from N,N-dimethyl acrylamide, N,N-diethyl acrylamide and N,N-dipropyl acrylamide.

According to a more particular aspect, in the composition (C1) that is the subject matter of the present invention, in the polyelectrolyte (P) as defined previously, the neutral monomer is N,N-dimethyl acrylamide.

According to a particular aspect, in the composition (C1) that is the subject matter of the present invention, the foaming surfactant comprises at least one compound of formula (II) as defined or at least one compound of formula (III) as defined above, or a mixture of at least one compound of formula (II) with at least compound of formula (III).

According to another particular aspect, the composition (C1) as defined previously is characterised in that the ratio by mass of foaming agent to crosslinked anionic polyelectrolyte (P) is between 1/10 and 40/1, more particularly between 1/1 and 40/1 and even more particularly between 4/1 and 40/1.

Partially or completely salified means, in particular for acid agents (A), partially or completely salified in the form of alkaline salts, such as the salt of sodium or the salt of potassium, alkaline-earth salts or ammonium salt.

α-hydroxy acid means in particular, in the definition of the composition (C1) that is the subject matter of the present invention, the compounds of formula formula (V):

R₇R₈C(OH)COOH (v)

in which R₇ and R₈ represent, independently of each other, a radical chosen from the hydrogen atom, the methyl radical, the —CH(OH)COOH radical, the —CH₂COOH radical, the —(CHOH)ₖ—CH₂OH radical in which k represents an integer between 1 and 6, or the phenyl radical.

By β-hydroxy acid we mean in particular, in the definition of the composition (C1) that is the subject matter of the present invention, 3-hydroxy butanoic acid, 3-hydroxy-3-methylbutanoic acid, carnitine or salicylic acid, and by the alcylated derivatives of salicylic acid of formula (VI):

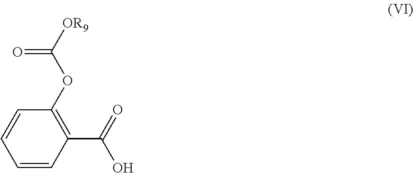

in which R₉ represents an aliphatic hydrocarbon radical, saturated or unsaturated, linear or branched, comprising 2 to 22 carbon atoms, such as for example 2-octanoyloxy-benzoic acid, 2-decanoyloxy benzoic acid or 2-dodecanoyloxy benzoic acid, and the corresponding salts thereof.

According to a particular aspect, the composition (C1) as previously defined is characterised in that the acid agent (A) is chosen from lactic, citric acid, glycolic acid, gluconic acid, tartric acid, malic acid or salicylic acid.

The composition (C1) that is the subject matter of the present invention is in particular in the form of a solution, an emulsion or a microemulsion with aqueous continuous phase, an emulsion or microemulsion with oily continuous phase, a gel or a foam, or in the form of an aerosol.

In general terms the composition (C1) that is the subject matter of the present invention comprises, in addition to said foaming surfactant and said acid agent (A) as defined previously, and water, adjuvants and/or additives normally used in the field of formulations for topical use, in particular cosmetics, dermocosmetiques, pharmaceuticals or dermo-pharmaceuticals. Among the adjuvants liable to be present in the compositions (C1) that are the subjects of the present invention, mention can be made of stabilising agents, film-forming compounds, solvents and co-solvents, hydrotropic agents, plasticisers, fats, oils, emulsifiers and co-emulsifiers, opacifiers, pearling agents, superfatting agents, sequestering agents, chelating agents, antioxidants, perfumes, preservatives, conditioners, bleaching agents intended for decolouring hair and skin, active principles intended to provide a treating action vis-à-vis the skin or hair, mineral fillers or pigments, particles procuring a visual effect or intended for the encapsulation of active agents, exfoliating particles, texture agents, optical brighteners or insect repellents.

Among the solvents and co-solvents that can be associated with the compositions (C1) that are the subjects of the present invention, mention will be made in particular of glycols, such as for example butylene glycol, hexylene glycol, caprylyl glycol or 1,2-octanediol, pentylene glycol or 1,2-pentanediol, pentylene glycol, monopropylene glycol, dipropylene glycol, isoprene glycol, butyldiglycol or the polyethylene glycols with a molecular weight of between 200 g·mol⁻¹ and 8000 g·mol⁻¹; alcohols such as for example ethanol or isopropanol; polyols such as for example glycerol, diglycerol, triglycerol, erythritol, xylitol, sorbitol or 2-methyl-1,3-propanediol; alkoxylated polyols.

Among the emulsifiers that can be associated with the compositions (C1) that are the subjects of the prevent invention, mention will be made in particular of fatty acids comprising 16 to 22 carbon atoms, ethoxylated fatty acids comprising 16 to 22 carbon atoms, fatty acid and sorbitol esters, polyglycerol esters comprising 16 to 22 carbon atoms, ethoxylated fatty alcohols comprising 16 to 22 carbon atoms, sucrose esters comprising 16 to 22 carbon atoms, alkylpolyglycosides the alkyl chain of which comprises 16 to 22 carbon atoms, sulfated and phosphated fatty alcohols or the mixtures of alkylpolyglycosides and fatty alcohols described in the French patent applications 2 668 080, 2 734 496, 2 756 195, 2 762 317, 2 784 680, 2 784 904, 2 791 565, 2 790 977, 2 807 435, 2 804 432, 2 830 774 and 2 830 445, the associations of emulsifying surfactants chosen from alkylpolyglycosides, associations of alkylpolyglycosides and fatty alcohols, polyglycerol or polyglycol esters or polyols such as polyhydroxy stearates of polyglycols or polyglycerols used in the French patent applications 2 852 257, 2 858 554, 2 820 316 and 2 852 258.

Among the opacifiers and/or pearling agents that can be associated with the compositions (C1) that are the subjects of the present invention, mention will be made in particular of sodium or magnesium palmitates or stearates or hydroxystearates, ethylene or polyethylene glycol monostearates or distearates, fatty alcohols, homopolymers and copolymers of styrene such as the styrene acrylate copolymer marketed under the name MONTOPOL™ OP1 by the company SEPPIC.

Examples of oils optionally present in the composition (C1) that is the subject of the present invention include:
mineral oils such as paraffin oil, vaseline oil, isoparaffins or mineral white oils;
oils of animal origin, such as squalene or squalane;
vegetable oils such as phytosqualane, sweet almond oil, copra oil, ricin oil, jojoba oil, olive oil, colza oil, groundnut oil, sunflower oil, wheatgerm oil, maize germ oil, soya oil, cotton oil, alfalfa oil, poppy oil, pumpkin oil, evening primrose oil, millet oil, barley oil, rye oil, sunflower oil, candleberry oil, passion-flower oil, hazelnut oil, palm oil, shea butter, apricot kernel oil, calophyllum oil, sisymbrium oil, avocado oil, calendula oil, or oils derived from flowers or vegetables;
ethoxylated vegetable oils;
synthetic oils such as fatty acid esters such as butyl myristate, propyl myristate, cetyl myristate, ispropyl palmitate, butyl stearate, hexadecyl stearate, isopropyl stearate, octyl stearate, isocetyl stearate, dodecyl oleate, hexyl laurate, propyleneglycol dicaprylate, esters derived from lanolic acid, such as isopropyl lanolate, isocetyl lanolate, monoglycerides, diglycerides and triglycerides of fatty acids such as glycerol triheptanoate, alkylbenzoates, hydrogenated oils, polyalphaolefins, polyolefins such as polyisobutene, synthetic isoalkanes such as isohexadecane, isododecane or perfluorinated oils, and
silicone oils such as dimethyl polysiloxanes, methylphenyl polysiloxanes, silicones modified by amines, silicones modified by fatty acids, silicones modified by alcohols, silicones modified by alcohols and fatty acids, silicones modified by polyether groups, modified epoxy silicones, silicones modified by fluorinated groups, cyclic silicones and silicones modified by alkyl groups.

As another fat optionally present in the formulation for which the method that is the subject matter of the present invention is implemented, mention can be made of fatty alcohols or fatty acids; waxes such as beeswax, carnauba wax, candelilla wax, ouricuri wax, Japan wax, cork fibre wax, sugar cane wax, paraffin waxes, lignite waxes, microcrystalline waxes, lanolin wax; ozokerite; polyethylene wax, silicone waxes; vegetable waxes; fatty alcohols and fatty acids solid at ambient temperature; glycerides solid at ambient temperature.

As examples of active principles other than α-hydroxy acids and β-hydroxy acids present in the composition (C1) that is the subject matter of the present invention, mention can be made of vitamins and the derivatives thereof, in particular esters thereof, such as retinol (vitamin A) and esters thereof (retinyl palmitate for example), ascorbic acid (vitamin C) and esters thereof (for example magnesium ascorbyl phosphate), derivates of ascorbic acid sugar (such as for example ascorbyl gluco side), tocopherol (vitamin E) and esters thereof (such as for example tocopherol acetate), vitamin B3 or B10 (niacinamide and derivatives thereof); compounds showing an action of lightening or depigmenting the skin, such as for example SEPIWHITE™ MSH, arbutin, kojic acid, hydroquinone, VEGEWHITE™, GATULINE™, SYNERLIGHT™, BIOWHITE™, PHYTOLIGHT™, DERMALIGHT™, CLARISKIN™, MELASLOW™ DERMAWHITE™, ETHIOLINE, MELAREST™, GIGAWHITETm, ALBATINE™, LUMISKIN™; the compounds showing a calming action such as SEPICALM™ S, allantoin and bisabolol; anti-inflammatory agents; compounds showing a hydrating action such as for example urea, hydroxyureas, polysaccharides, glycerol, polyglycerols, AQUAXYL™, glycerol glucoside; extracts of polyphenols such as for example grape extracts, pine extracts, wine extracts, olive extracts; compounds showing a slimming or lipolytic action such as cafeine or derivatives thereof, ADIPOSLIM™, ADIPOLESS™; N-acylated proteins; N-acylated peptides such as for example MATRIXIL™; N-acylated amino acids; partial hydrolysates of N-acylated proteins; amino acids; peptides; total hydrolysates of proteins; soya extracts, for example Raffermine™; wheat extracts, for example TENSINE™ or GLIADINE™; plant extracts, such as plant extracts rich in tanins, plant extracts rich in isoflavones or plant extracts rich in terpenes; extracts of fresh water or marine algae; marine extracts in general such as corals; essential waxes; bacterial extracts; minerals such as derivatives of calcium, magnesium, copper, cobalt, zinc, lithium or manganese; silver or gold salts; ceramides; phospholipids; compounds showing an antimicrobial action or a purifying action, such as for example LIPACIDE™ CBG, LIPACIDE™ UG, SEPICONTROL™ A5; OCTOPIROX™ or SENSIVA™ SC50; compounds showing an energising or stimulating property such as SEPITONIC™ M3 or Physiogenyl™, panthenol and derivatives thereof such as SEPICAP™ MP; anti-ageing active agents such as SEPILIFT™ DPHP, LIPACIDE™ PVB, SEPIVINOL™, SEPIVITAL™, MANOLIVA™, PHYTO-AGE™, TIMECODE™; SURVICODE™; anti-photoageing active agents; active agents protecting the integrity of the dermo-epidermal junction; active agents increasing the synthesis of components of the extracellular matrix such as for example collagen, elastins, glycosaminoglycanes; active agents acting favourably on chemical cell communication such as cytokines or physical cell communication such as integrines; active agents creating a sensation of “heating” on the skin, such as activators of skin microcirculation (such as for example nicotinates) or products creating a sensation of “coolness” on the skin (such as for example menthol and derivatives thereof); active agents improving skin microcirculation, for example veinotonics; draining active agents; active agents with a decongestioning purpose such as for example extracts of ginko biloba, ivy, Indian horse chestnut, bamboo, ruscus, butcher's broom, Centalla asiatica, wrack, rosemary or willow.

Another subject matter of the invention is a method for preparing a composition (C1) as defined previously, characterised in that it comprises:

a step (a) of mixing, under agitation, said foaming surfactant, said acid agent (A) and water,

a step (b) of mixing under agitation the combination prepared at step (a), with said crosslinked anionic polyelectrolyte (P).

Steps a) and b) of the method that is the subject matter of the present invention are generally conducted at a temperature of between 20° C. and 60° C., more particularly between 20° C. and 40° C., under mechanical agitation by means of an agitator provided with a blade of the anchor type, at an agitation speed of between 50 revolutions/minute and 600 revolutions/minute, more particularly between 50 revolutions/minute and 300 revolutions/minute.

According to a last aspect, the subject matter of the invention is the use of a composition (C1) as defined previously for the cleaning of and/or makeup removal from the skin of the face and/or body and more particularly use as defined previously for the cosmetic treatment of acne and/or blackheads and/or comedones.

The following examples illustrate the invention without however limiting same.

### EXAMPLE 1

### 1.1. Preparation of a terpolymer of ammonium 2-methyl 2-[(1-oxo 2-propenyl)amino]1-propanesulfonate, N,N-dimethyl acrylamide and tetraethoxylated lauryl methacrylate [AMPS/DMAM/MAL(40E) 77.4/19.2/3.4 molar], crosslinked with trimethylol propanetriacrylate (TMPTA) [example according to the invention]

592 g of an aqueous solution at 15% by mass of ammonium 2-methyl 2-[(1-oxo 2-propenyl)amino]1-propanesulfonate in a tert-butanol/water mixture (97.5/2.5 by volume), 10.1 g of N,N-dimethyl acrylamide, 4.2 g of tetraethoxylated lauryl methacrylate and 0.75 g of trimethylol propanetriacrylate are loaded into a reactor maintained at 25° C. under agitation.

After sufficient time to achieve good homogenisation of the solution, the latter is deoxygenated by bubbling nitrogen heated to 70° C. 0.42 g of dilauroyl peroxide is then added and the reaction medium is then maintained for approximately 60 minutes at 70° C. and then 2 hours at 80° C.

After cooling, the powder that formed during polymerisation is filtered and dried in order to obtain the desired product, hereinafter referred to as: Polyelectrolyte 1.

### 1.2. Preparation of a terpolymer of ammonium 2-methyl 2-[(1-oxo 2-propenyl)amino]1-propanesulfonate, 2-hydroxyethylacrylate and tetraethoxylated lauryl methacrylate [AMPS/HEA/MAL(40E) 77.4/19.2/3.4 molar], crosslinked with trimethylol propanetriacrylate (TMPTA) [comparative example]

Using the operating conditions of the method described in example 1.1 above, the necessary quantity by mass of aqueous solution at 15% by mass of ammonium 2-methyl 2-[(1-oxo 2-propenyl)amino]1-propanesulfonate in a tert-butanol/water mixture (97.5/2.5 by volume) is loaded into a reactor maintained at 25° C. under agitation so as to introduce 77.4 molar equivalents of ammonium 2-methyl 2-[(1-oxo 2-propenyl)amino]1-propanesulfonate, the necessary quantity by mass of 2-hydroxyethylacrylate so as to introduce 19.2 molar equivalents of 2-hydroxyethylacrylate, the necessary quantity by mass of tetraethylated lauryl methacrylate so as to introduce 3.4 molar equivalents of tetraethoxylated lauryl methacrylate, and the necessary quantity by mass of trimethylol propanetriacrylate so as to obtain the same molar proportion of trimethylol propanetriacrylate as in example 1.1.

After sufficient time to achieve good homogenisation of the solution, the latter is deoxygenated by bubbling nitrogen heated to 70° C. 0.42 g of dilauroyl peroxide is then added and the reaction medium is then maintained for approximately 60 minutes at 70° C. and then 2 hours at 80° C.

After cooling, the powder that formed during polymerisation is filtered and dried in order to obtain the desired product, hereinafter referred to as: Polyelectrolyte 2.

### 1.3. Preparation of a copolymer of ammonium 2-methyl 2-[(1-oxo 2-propenyl)amino]1-propanesulfonate and tetraethoxylated lauryl methacrylate [AMPS/MAL(40E) 95/5 molar], crosslinked with trimethylol propanetriacrylate (TMPTA) [comparative example]

Using the operating conditions of the method described in example 1.1 above, the necessary quantity by mass of aqueous solution at 15% by mass of ammonium 2-methyl 2-[(1-oxo 2-propenyl)amino]1-propanesulfonate in a tert-butanol/water mixture (97.5/2.5 by volume) is loaded into a reactor maintained at 25° C. under agitation so as to introduce 95 molar equivalents of ammonium 2-methyl 2-[(1-oxo 2-propenyl)amino]1-propanesulfonate, the necessary quantity by mass of tetraethylated lauryl methacrylate so as to introduce 5 molar equivalents of tetraethoxylated lauryl methacrylate, and the necessary quantity by mass of trimethylol propanetriacrylate so as to obtain the same molar proportion of trimethylol propanetriacrylate as in example 1.1.

After sufficient time to achieve good homogenisation of the solution, the latter is deoxygenated by bubbling nitrogen heated to 70° C. 0.42 g of dilauroyl peroxide is then added and the reaction medium is then maintained for approximately 60 minutes at 70° C. and then 2 hours at 80° C.

After cooling, the powder that formed during polymerisation is filtered and dried in order to obtain the desired product, hereinafter referred to as: Polyelectrolyte 3.

### 1.4. Preparation of a copolymer of ammonium 2-methyl 2-[(1-oxo 2-propenyl)amino]1-propanesulfonate and 2-hydroxyethylacrylate [AMPS/HEA 90/10 molar], crosslinked with trimethylol propanetriacrylate (TMPTA) [comparative example]

Using the operating conditions of the method described in example 1.1 above, the necessary quantity by mass of aqueous solution at 15% by mass of ammonium 2-methyl 2-[(1-oxo 2-propenyl)amino]1-propanesulfonate in a tert-butanol/water mixture 97.5/2.5 by volume) is loaded into a reactor maintained at 25° C. under agitation so as to introduce 90 molar equivalents of ammonium 2-methyl 2-[(1-oxo 2-propenyl)amino]1-propanesulfonate, the necessary quantity by mass of 2-hydroxyethylacrylate so as to introduce 10 molar equivalents of 2-hydroxyethylacrylate, and the necessary quantity by mass of trimethylol propanetriacrylate so as to obtain the same molar proportion of trimethylol propanetriacrylate as in example 1.1.

After sufficient time to achieve good homogenisation of the solution, the latter is deoxygenated by bubbling nitrogen heated to 70° C. 0.42 g of dilauroyl peroxide is then added and the reaction medium is then maintained for approximately 60 minutes at 70° C. and then 2 hours at 80° C.

After cooling, the powder that formed during polymerisation is filtered and dried in order to obtain the desired product, hereinafter referred to as: Polyelectrolyte 4.

### EXAMPLE 2

### A—Preparation of Cleaning Compositions According to the Invention, Comprising Polyelectrolyte 1, and Comparative Formulations

Water is poured into a beaker, at 20°, in the proportions by mass indicated in table 1, and then, gradually and under mechanical agitation at 50 revolutions/minute, the foaming surfactant, and then gradually the thickening agent, and then the neutralising agent (acidic or basic depending on the formulations) so as to obtain the desired pH. The formulations (F1), (F11), (F12) and (F13) according to the invention and the comparative formulations (F2) to (F10) are obtained.

TABLE 1

| | (F1) | (F2) | (F3) | (F4) |
|---|---|---|---|---|
| Sodium lauryl ether sulphate (2.2 | 44.3% | 44.3% | 44.3% | 44.3% |
| OE) (27.1% M.A.) | | | | |
| Amonyl ™ 380 BA⁽¹⁾ (28.9% | 10.4% | 10.4% | 10.4% | 10.4% |
| M.A.) | | | | |
| Amonyl ™ 675 SB⁽²⁾ (38.44% | 0% | 0% | 0% | 0% |
| M.A.) | | | | |
| Water | 44.3% | 43.6% | 42.4% | 42.5% |
| Lactic acid in solution at 45% | 0.2% | 0.2% | 0% | 0% |
| Citric acid in solution at 10% | 0% | 0% | 0% | 0.5% |
| Triethanolamine in solution at 50% | 0% | 0% | 0.6% | 0% |
| Soda | 0% | 0% | 0% | 0% |
| Polyelectrolyte 1 | 0.8% | 0% | 0% | 0% |
| NaCl | 0% | 1.5% | 0% | 0% |
| Capigel ™ 98⁽³⁾ | 0% | 0% | 2.3% | 2.3% |
| Gum xanthan | 0% | 0% | 0% | 0% |
| pH measured at 20° C. | 5.2 | 4.7 | 7.0 | 5.5 |

| | (F5) | (F6) | (F7) |
|---|---|---|---|
| Sodium lauryl ether sulphate (2.2 | 44.3% | 35.0% | 35.0% |
| OE) (27.1% M.A.) | | | |
| Amonyl ™ 380 BA⁽¹⁾ (28.9% M.A.) | 10.4% | 0% | 0% |
| Amonyl ™ 675 SB⁽²⁾ (38.44% M.A.) | 0% | 5% | 5% |
| Water | 44.0% | 59.1% | 56.6% |
| Lactic acid in solution at 45% | 0.1% | 0% | 0% |
| Citric acid in solution at 10% | 0% | 0% | 0% |
| Triethanolamine in solution at 50% | 0% | 0% | 0% |
| Soda | 0% | 0.1% | 0.1% |
| Polyelectrolyte 1 | 0% | 0.8% | 3.3% |
| NaCl | 0% | 0% | 0% |
| Capigel ™ 98(3) | 0% | 0% | 0% |
| Gum xanthan | 1.2% | 0% | 0% |
| pH measured at 20° C. | 5.5 | 7.2 | 7.3 |
| | (F8) | (F9) | (F10) |
| Sodium lauryl ether sulphate (2.2 | 44.3% | 44.3% | 44.3% |
| OE) (27.1% M.A.) | | | |
| Amonyl ™ 380 BA⁽¹⁾ (28.9% M.A.) | 10.4% | 10.4% | 10.4% |
| Amonyl ™ 675 SB⁽²⁾ (38.44% M.A.) | 0% | 0% | 0% |
| Water | 43.4% | 42.9% | 42.9% |
| Lactic acid in solution at 45% | 0.4% | 0.4% | 0.4% |
| Citric acid in solution at 10% | 0% | 0% | 0% |
| Triethanolamine in solution at 50% | 0% | 0% | 0% |
| Soda | 0% | 0% | 0% |
| Polyelectrolyte 2 | 1.5% | 0% | 0% |
| Polyelectrolyte 3 | 0% | 2.0% | 0% |
| Polyelectrolyte 4 | 0% | 0% | 2.0% |
| NaCl | 0% | 0% | 0% |
| Capigel ™ 98⁽³⁾ | 0% | 0% | 0% |
| Gum xanthan | 0% | 0% | 0% |
| pH measured at 20° C. | 4.2 | 4.9 | 4.8 |
| | (F11) | (F12) | (F13) |
| Sodium lauryl ether sulphate (2.2 | 0% | 0% | 0% |
| OE) (27.1% M.A.) | | | |
| Amonyl ™ 380 BA⁽¹⁾ (28.9% M.A.) | 10.4% | 10.4% | 0% |
| Oramix ™ 10⁽⁴⁾ (54.24% M.A.) | 22.73% | 0% | 22.73% |
| Proteol ™APL⁽⁵⁾ (22.4% M.A.) | 0% | 55.0% | 0% |
| Water | 64.57% | 32.2% | 74.87% |
| Lactic acid in solution at 45% | 0.3% | 0.4% | 0.4% |
| Citric acid in solution at 10% | 0% | 0% | 0% |
| Triethanolamine in solution at 50% | 0% | 0% | 0% |
| Soda | 0% | 0% | 0% |
| Polyelectrolyte 1 | 2.0% | 2.0% | 2.0% |
| NaCl | 0% | 0% | 0% |
| Capigel ™ 98⁽³⁾ | 0% | 0% | 0% |
| Gum xanthan | 0% | 0% | 0% |
| pH measured at 20° C. | 5.1 | 6.3 | 5.1 |

% M.A.: % by mass of active material.
⁽¹⁾AMONYL ™ 380 BA is a cocoamidopropyl betaine, marketed by the company SEPPIC.
⁽²⁾AMONYL ™ 675SB is a cocoamidopropyl hydroxy sultaine, marketed by the company SEPPIC.
⁽³⁾CAPIGEL ™ 98 is an acrylate copolymer of the HASE type, marketed by the company SEPPIC.
⁽⁴⁾ORAMIX ™ NS 10 is a foaming composition comprising octanoylpolyglucoside and decylpolyglucoside marketed by the company SEPPIC.
⁽⁵⁾PROTEOL ™ APL is a foaming composition comprising a sodium salt of cocoyl amino acids derived from apple juice, marketed by the company SEPPIC.

### B—Demonstration of the Properties and Characteristics of the Cleaning Formulations According to the Invention Compared with Those of the Prior Art

The formulations (F1) to (F13) previously prepared are then evaluated as follows:
Measurement of their pH at 20° C.;
Measurement of their viscosity at 20° C. by means of a viscosimeter of the Brookfield LVT type at a speed of 6 revolutions/minute, fitted with the appropriate spindle;
Visual evaluation of their appearance;
Evaluation of the size and stability of the foam generated by each of them according to the experimental protocol disclosed below.

Experimental Protocol for Evaluating the Size and Stability of the Foam Bubbles Generated in Formulations (F1) to (F13)

i) Preparation of a Foaming Solution (S)

For each of the formulations (F1) to (F13) to be tested, an aqueous solution (S) of 500 cm³ at 1% by mass of active material of the formulation to be tested, in demineralised water enriched with 3 millimoles of calcium ions (corresponding to the preparation of a water at 30° of calcic hardness in accordance with NFT 73-047).

ii) Preparation of the Test Solution

For each of the formulations (F1) to (F13) to be tested, 25 cm³ of the corresponding solution (S) is taken off, in a high-shaped 100 cm³ beaker, which is then placed in a thermostatically-controlled water bath for 30 minutes, in order to reach a temperature of 40° C.+/−1° C.

iii) Swelling of the Test Solution

When the test solution (S) is stabilised at a temperature of 40° C., it is agitated in a beaker by means of an agitator of the Rayneri type provided with a centripetal turbine of suitable size at a speed of 3000 revolutions/minute, for a period of two minutes so as to generate a foam.

iv) Observation of the Foam Generated by the Test Solution

The foam formed is decanted into a Petri dish (5.3 cm diameter×1.2 cm high) to overflowing and then spread over the entire surface of the dish. This Petri dish is placed under the lens of an electron microscope of make NIKON OPTIPHOP-2, provided with a NIKON DXM 1200 digital camera and associated with a computer equipped with the NIKON ACT-1 software; the magnification being ×40. Focusing is carried out and a first photograph is taken coinciding with the triggering of a chronometer. New photographs are then taken after a period of 10 minutes and 20 minutes following the triggering of the chronometer.

v) Expression of the Results

The operations described in paragraphs ii) to iv) above are reproduced for each of formulations (F1) to (F13) so as to have a significant statistical population. For each photograph taken for a given formulation and at a given time, the experimenter identifies the total number of bubbles present on the photograph (N_{T}) and measures, for each of the bubbles, the corresponding diameter, by means of the micrometric scale present and made available by the NIKON ACT-1 software.

The experimenter then records:
The number of bubbles with a diameter less than or equal to 150 micrometers (N1) for each of the photographs relating to a given formulation;
The number of bubbles with a diameter greater than 150 micrometres and less than or equal to 450 micrometres (N2);
The number of bubbles with a diameter greater than 450 micrometres (N3).

The experimenter then calculates, for each photograph, the following ratios:

R1=(N1/NT)×100

R2=(N2/NT)×100

R3=(N3/NT)×100

The experimenter calculates the average of the ratios R1, R2 and R3 at t=0, called respectively R1m₀, R2m₀ and R3mₒ, then the average of the ratios R1, R2 and R3 at t=10 minutes, called respectively R1m₁₀, R2m₁₀ and R3m₁₀, and the average of the ratios R1, R2 and R3 at t=20 minutes, called respectively R1m₂₀, R2m₂₀ and R3m₂₀.

The results of these evaluations are set out in the following table 2.

TABLE 2

| | (F1) | (F2) | (F3) | (F4) |
|---|---|---|---|---|
| pH | 5.2 | 4.7 | 7.0 | 5.5 |
| Viscosity (in mPas) | 4529 | 9800 | 6200 | 430 |
| Appearance (visual) | Homogeneous | Homogeneous | Homogeneous | Homogeneous |
| | liquid | liquid | liquid | liquid |

Distribution of the diameters of the foam bubbles at T = 0

| | | | | |
|---|---|---|---|---|
| R1m₀ | 91.0% | 73.5% | 83.9% | 87.8% |
| R2m₀ | 9% | 26.5% | 16.1% | 12.2% |
| R3m₀ | 0% | 0% | 0% | 0% |

Distribution of the diameters of the foam bubbles at T = 10 minutes

| | | | | |
|---|---|---|---|---|
| R1m₁₀ | 78% | 50.0% | 50.0% | 53.3% |
| R2m₁₀ | 22% | 50.0% | 47.1% | 44.4% |
| R3m₁₀ | 0% | 0% | 2.9% | 2.3% |

Distribution of the diameters of the foam bubbles at T = 20 minutes

| | | | | |
|---|---|---|---|---|
| R1m₂₀ | 76.5% | 35.3% | 52.9% | 50% |
| R2m₂₀ | 23.5% | 41.7% | 29.4% | 40.9% |
| R3m₂₀ | 0% | 17.6% | 17.7% | 9.1% |

| | (F5) | (F6) | (F7) |
|---|---|---|---|
| pH | 5.5 | 7.2 | 7.3 |
| Viscosity (in mPas) | 6400 | 70 | 270,000 |
| Appearance (visual) | Slightly viscous | Homogeneous | Compact |
| | heterogeneous | liquid | non-liquid |
| | liquid | | |

Distribution of the diameters of the foam bubbles at T = 0

| | | | |
|---|---|---|---|
| R1m₀ | 85.5% | 75.4% | 95.4% |
| R2m₀ | 14.5% | 24.6% | 4.5% |
| R3m₀ | 0% | 0% | 0% |

Distribution of the diameters of the foam bubbles at T = 10 minutes

| | | | |
|---|---|---|---|
| R1m₁₀ | 61.7% | 51.3% | 62.0% |
| R2m₁₀ | 34.0% | 46.3% | 62.0% |
| R3m₁₀ | 4.3% | 2.4% | 0.0% |

Distribution of the diameters of the foam bubbles at T = 20 minutes

| R1m₂₀ | 31.8% | 34.8% | 48.5% |
|---|---|---|---|
| R2m₂₀ | 50.0% | 43.5% | 33.3% |
| R3m₂₀ | 18.2% | 21.7% | 18.2% |
| | (F8) | (F9) | (F10) |
| pH | 4.2 | 4.9 | 4.8 |
| Viscosity (in mPas) | 3300 | 80 | 100 |
| Appearance (visual) | Homogeneous | Homogeneous | Cloudy |
| | liquid | liquid | homogeneous |
| | | | liquid |

Distribution of the diameters of the foam bubbles at T = 0

| | | | |
|---|---|---|---|
| R1m₀ | 97.3% | n.m | n.m |
| R2m₀ | 2.7% | n.m | n.m |
| R3m₀ | 0% | n.m | n.m |

Distribution of the diameters of the foam bubbles at T = 10 minutes

| R1m₁₀ | 58.7% | n.m | n.m |
|---|---|---|---|
| R2m₁₀ | 39.1% | n.m | n.m |
| R3m₁₀ | 2.2% | n.m | n.m |
| | (F11) | (F12) | (F13) |
| pH | 5.1 | 6.3 | 5.1 |
| Viscosity (in mPas) | 3885 | 2010 | 7460 |
| Appearance (visual) | Homogeneous | Homogeneous | Homogeneous |
| | liquid | liquid | liquid |

Distribution of the diameters of the foam bubbles at T = 0

| | | | |
|---|---|---|---|
| R1m₀ | 94.9% | 95.5% | 98.8% |
| R2m₀ | 5.1% | 4.5% | 5.1% |
| R3m₀ | 0% | 0% | 0% |

Distribution of the diameters of the foam bubbles at T = 10 minutes

| | | | |
|---|---|---|---|
| R1m₁₀ | 72.9% | 93.6% | 96.1% |
| R2m₁₀ | 27.1% | 6.4% | 3.9% |
| R3m₁₀ | 0% | 0% | 0% |

n.m.: not measured.

### 3) Analysis of the Results

The results are judged to be satisfactory when the appearance of the formulation is liquid, the viscosity is between 2,000 mPa·s and 30,000 mPa·s, when, in the foam generated by the formulation, the proportion in terms of the number of bubbles with a mean diameter of less than or equal to 150 micrometres after 10 minutes following the swelling (measured by R1m₁₀) is greater than 70%.

The formulation (F1) according to the invention is characterised by a liquid appearance, a viscosity measured at 4529 mPa·s and the foam formed by (F1) shows a proportion in terms of the number of bubbles with a mean diameter less than or equal to 150 micrometres after 10 minutes following swelling equal to 78%. It is also observed that this foam is very stable since, after 20 minutes following the swelling, the proportion in terms of the number of bubbles with a mean diameter less than or equal to 150 micrometres is equal to 76.5%.

For the foams obtained from the formulations (F6) and (F7), which are disclosed and taught by the international publication WO 2011/030044, and which are characterised by a pH value greater than 7.0, a proportion in terms of the number of bubbles with a mean diameter less than or equal to 150 micrometres after 10 minutes following the swelling is observed at respectively 51.3% for (F6) and 62% for (F7). In the foam generated by the formulation (F6), a proportion in terms of the number of bubbles with a mean diameter greater than 450 micrometres after 10 minutes following swelling should also be noted to be equal to 2.4%.

For the foam obtained from the formulation (F2), thickened by the use of 1.5% of sodium chloride, a proportion in terms of the number of bubbles with a mean diameter less than or equal to 150 micrometres after 10 minutes following the swelling of 50% is observed.

For the foams formed by the formulations (F3) and (F4), thickened by the use of Capigel™ 98, a polymer of the HASE type, a proportion in terms of the number of bubbles with a mean diameter of less than or equal to 150 micrometres after 10 minutes following the swelling is observed equal respectively to 50% and 53.3%, as well as a non-zero proportion of bubbles with mean diameters greater than 450 micrometres after 10 minutes following swelling.

For the foam obtained from the formulation (F5), thickened by the use of 2.25% of gum xanthan, a proportion in terms of the number a proportion in terms of the number of bubbles with a mean diameter of less than or equal to 150 micrometres after 10 minutes following swelling of 61.7% is observed and a proportion equal to 4.3% of bubbles with a mean diameter greater than 450 micrometres after 10 minutes following swelling.

The formulation (F8) comprising the Polyelectrolyte 2 is characterised by a homogeneous liquid appearance and a viscosity measured at 3300 mPa·s, and the foam formed by (F8) shows a proportion in terms of the number of bubbles with a mean diameter less than or equal to 150 micrometres after 10 minutes following swelling equal to 58.7%.

The formulation (F9) comprising the Polyelectrolyte 3 is characterised by a homogeneous liquid appearance and a viscosity measured at 80 mPa·s. The formulation (F9) does not make it possible to obtain the required viscosity level, namely a minimum viscosity of 2,000 mPa·s.

The formulation (F10) comprising the Polyelectrolyte 4 is characterised by a homogeneous liquid appearance and a viscosity measured at 100 mPa·s. The formulation (F10) does not make it possible to obtain the required viscosity level, namely a minimum viscosity of 2,000 mPa·s.

The formulations (F11), (F12) and (F13) according to the invention are characterised by a homogeneous liquid appearance and a measured viscosity greater than 2,000 mPa·s, and the foams formed by the formulations (F11), (F12) and (F13) show a proportion in terms of the number of bubbles with a mean diameter less than or equal to 150 micrometres after 10 minutes following swelling greater than 70%.

A comparison of these measurements indicates clearly that the improvement in foaming properties of a cleaning formulation for topical use, comprising at least one foaming surfactant and having a pH greater than or equal to 4.0 and less than or equal to 6.5, is afforded by the implementation of the method according to the invention, which consists of incorporating, in said cleaning formulation, an effective quantity of a crosslinked anionic polyelectrolyte (P) as described previously.

1. Method for improving the foaming properties of a cleaning formulation for topical use, with a pH greater than or equal to 4.0 and less than or equal to 6.5 and comprise at least one foaming surfactant, said method being characterised in that an effective quantity of a crosslinked anionic polyelectrolyte (P) derived from polymerisation is incorporated in said cleaning formulation for topical use, in the presence of at least one crosslinking agent and at least one monomer having a strong acid function, said monomer being 2-methyl 2-[(1-oxy 2-propenyl)amino]1-propanesulfonic acid, partially or completely salified, with at least one neutral monomer chosen from the N,N-dialkyl acrylamides, in which each of the alkyl groups comprises between one and four carbon atoms, and at least one monomer of formula (I): 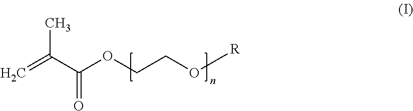
in which R represents a linear or branched alkyl radical comprising from eight to twenty carbon atoms and n represents a number greater than or equal to one and less than or equal to twenty.

2. Method as defined in claim 1, characterised in that said crosslinked anionic polyelectrolyte (P) comprises, for 100% molar:
from 20% molar to 80% molar of monomeric units derived from the monomer comprising a partially or completely salified strong acid function;
from 15% molar to 75% molar of monomeric units derived from a neutral monomer chosen from the N,N-dialkyl acrylamides, wherein each of the alkyl groups comprises between one and four carbon atoms;
from 0.5% molar to 5% molar of monomeric units derived from a monomer of formula (I) as defined previously.

3. Method as defined in claim 1, characterised in that, in said crosslinked anionic polyelectrolyte (P), said neutral monomer is N,N-dimethyl acrylamide.

4. Method as defined in claim 1, characterised in that, in said crosslinked anionic polyelectrolyte (P), said monomer of formula (I) is tetraethoxylated lauryl methacrylate.

5. Method as defined in claim 1, characterised in that said crosslinked anionic polyelectrolyte (P) is a terpolymer of 2-methyl 2-[(1-oxo 2-propenyl)amino]1-propanesulfonic acid partially salified in ammonium salt form, N,N-dimethyl acrylamide and tetraethoxylated lauryl methacrylate, crosslinked with trimethylol propanetriacrylate.

6. Method as defined in claim 1, characterised in that said crosslinked anionic polyelectrolyte (P) comprises, for 100% molar:
from 60% molar to 80% molar of monomeric units derived from 2-methyl 2-[(1-oxo 2-propenyl)amino]1-propanesulfonic acid partially salified in ammonium form;
from 15% molar to 39.5% molar of monomeric units derived from N,N-dimethyl acrylamide; and
from 0.5% molar to 5% molar of monomeric units derived from tetraethoxylated lauryl methacrylate.

7. Method as defined in claim 1, for which the cleaning formulation for topical use comprises at least one anionic foaming surfactant of formula (II):
R₂—O—(CH₂—CH₂—O)ₚSO₃—X (II)
in which R₂ represents an aliphatic hydrocarbon radical, saturated or unsaturated, linear or branched, comprising 6 to 22 carbon atoms, p represents a decimal number between 1 and 10, preferably between 2 and 4, and X represents the cation of an alkaline metal or alkaline-earth metal, the ammonium ion, the hydroxyethyl ammonium ion, the tris-hydroxyethyl ammonium ion or a mixture of compounds of formula (II).

8. Method as defined in claim 1, for which the cleaning formulation for topical use comprises at least one amphoteric foaming surfactant of formula (III):
R₃—C(O)—NH(CH₂)_{q}—N⁺(R₄)(R₅)—(CH₂)ₛ—CO₂ (III)
in which R₃ represents an aliphatic hydrocarbon radial, saturated or unsaturated, linear or branched, comprising 7 to 21 carbon atoms, R₄ and R₅ represent independently of each other an aliphatic radical, saturated or unsaturated, linear or branched, optionally substituted with a hydroxyl group, comprising 1 to 4 carbon atoms, q represents an integer number between 2 and 6, and s represents an integer number equal to 1 or 2, or a mixture of compounds of formula (III).

9. Method as defined in claim 1, for which the cleaning formulation for topical use comprises a mixture of at least one compound of formula (II) as defined previously with at least one compound of formula (III) as defined previously.

10. Method as defined in claim 1, for which the cleaning formulation for topical use comprises as least one compound of formula (IV):
R₆—O—(S)_{y}—H (IV)
in which:
y is a decimal number between 1.05 and 2,
S represents the remainder of a reducing sugar chosen from glucose, xylose or arabinose,
R₆ represents a radical chosen from the n-octyl, n-decyl, n-dodecyl, n-tetradecyl or n-hexadecyl radicals, or a mixture of compounds of formula (IV).

11. Method as defined in claim 1, characterised in that the ratio by mass of foaming surfactant to crosslinked anionic polyelectrolyte (P) is between 1/10 and 40/1.

12. Composition (C1) characterised in that it comprises, for 100% of its mass:
From 0.05% to 2% by mass of at least one crosslinked anionic polyelectrolyte (P) derived from the polymerisation of at least one monomer having a strong acid function, said monomer being 2-methyl 2-[(1-oxo 2-propenyl)amino]1-propanesulfonic acid partially or totally salified, with at least one neutral monomer chosen from the N,N-dialkyl acrylamides, wherein each of the alkyl groups comprises between one and four carbon atoms, and at least one monomer of formula (I): 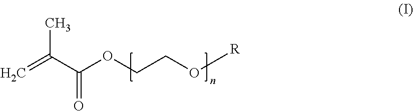
in which R represents a linear or branched alkyl radical comprising from eight to twenty carbon atoms and n represents a number greater than or equal to one and less than or equal to twenty, in the presence of at least one crosslinking agent;
from 10% to 50% by mass of at least one foaming surfactant selected from elements of the group formed by the anionic surfactants, non-anionic surfactants and amphoteric surfactants.
from 0.01% to 10% by mass of at least one acid agent (A) selected from the group consisting of the α-hydroxy acids and β-hydroxy acids, free, partially or completely salified.
from 89.94% to 38% by mass of water,
and in that the pH thereof is greater than or equal to 4.0 and less than or equal to 6.5.

13. Composition (C1) as defined in claim 12, characterised in that the ratio by mass of foaming surfactant to crosslinked anionic polyelectrolyte (P) is between 1/10 and 40/1.

14. Composition (C1) as defined in claim 12, characterised in that the acid agent (A) is selected from the elements in the group consisting of lactic acid, citric acid, glycolic acid, gluconic acid, tartric acid, malic acid and salicylic acid.

15. Method of cleaning and/or removing makeup from the skin of the face and/or body, which comprises applying an effective amount of the composition of claim 12 to the skin.

16. Method as defined in claim 2, characterised in that, in said crosslinked anionic polyelectrolyte (P), said neutral monomer is N,N-dimethyl acrylamide.

17. Composition (C1) as defined in claim 13, characterised in that the acid agent (A) is selected from the elements in the group consisting of lactic acid, citric acid, glycolic acid, gluconic acid, tartric acid, malic acid and salicylic acid.
